(19) United States
(12) Patent Application Publication
(10) Pub. No.: US 2020/0068933 A1
(43) Pub. Date: Mar. 5, 2020

(54) PLANT PRESS USING SHEARING FORCE

(71) Applicant: H.T.P. Science Co. L.L.C., Pocono Summit, PA (US)

(72) Inventor: Gordon Chiu, Summit, NJ (US)

(73) Assignee: H.T.P. Science Co. L.L.C., Pocono Summit, PA (US)

(21) Appl. No.: 16/118,584

(22) Filed: Aug. 31, 2018

(51) Int. Cl. A23L 33/105; A23L 27/10; B01D 11/02

(52) U.S. Cl. CPC A23L 33/105; A61K 2236/30; B01D 11/0246; A23L 27/115

(57) ABSTRACT

A plant press including a container having a cavity for receiving a plant and a plunger disposed within the cavity of the container. The plunger being translatable in a direction transverse to a longitudinal axis of the cavity and adapted to create a shearing force.

### BACKGROUND OF THE INVENTION

The present disclosure relates generally to a plant press and, more particularly, to a plant press having a chamber provided with a plunger for application of a shearing force.

A plant extract is a substance or an active with desirable properties that is removed from a tissue of a plant, usually by treating it with a solvent. Extraction success often depends on the extraction technique, input parameters and the exact nature of the specific plant parts. The most common factors affecting extraction processes are matrix properties of the plant part, solvent, temperature, pressure and time.

Plant extracts are used in a variety of sectors for different purposes, for example, as antioxidants and texturizers within the foodstuff industry, as chemical replacers in processing aids, for therapeutic benefits within the pharmaceutical industry, and for preventive and/or curative benefits within the cosmetic industry.

One such plant extract is essential oils, which are produced in the cells of aromatic plants and held in specialized glands. They are released from the plant and collected most often through steam distillation, a method of separating components based on the differences in volatile constituents in a heated mixture and bubbling the steam through the plant material. Alternatively, the essential oils may be “cold pressed” or extracted using high mechanical pressure to literally squeeze the oil from the plant material.

There are also a plethora of extraction techniques for medicinal plants, such as, maceration, decoction, percolation, soxhlet, and aqueous alcoholic extraction by fermentation. Again, the technique by which extraction is may be performed is often dependent upon the specific plant parts.

Another type of plant extract is hashish. Hashish or “hash” is a consumable compress of purified psychoactive resins from a cannabis plant. The highest concentration of these resins is found in the buds of the plant, which are typically smoked and consumed for medicinal or recreational purposes via inhalation. Around ten percent of the resins, however, are located in the leaves and stems (collectively “skuff”) of the cannabis plant, which is ordinarily discarded due to its unpleasantness as a smoked or eaten consumable.

As societal stigma surrounding cannabis use declines and cannabis use continues to be decriminalized at the state level, it is becoming more and more desirable to extract to the psychoactive resins from cannabis skuff so that the same may be recreationally or medicinally consumed.

Various mechanisms currently exist for extracting the psychoactive cannabis resins. One popular method is known as flat screening. Flat screening is typically accomplished by manually rubbing skuff over a fine steel or silk screen that is placed over a mirror or glass. After contacting the skuff with the screen, the resins pass through the screen and may be collected on the mirror or glass while the skuff remains on the screen. While flat screening is a simple process, the quality and amount of resins collected is dependent on the skill of the screener. As a result, flat screening frequently results in hash with contaminants or impurities.

A plant drying press is yet another mechanism by which resins may be extracted. In a typical drying press, skuff is clamped between a pair of vapor permeable pads through which microwave energy and/or heat is applied. While drying presses are an efficient method of extracting the resin, the application of microwave energy and/or heat to the plant often shocks and denatures the plant's proteins, thereby diminishing the desirable properties of the extract.

There therefore is a need for an efficient and automated apparatus for extracting a plant extract from a variety of plants. Moreover, the apparatus should permit removal of the extract with minimal impurities and be performed in a manner that maintains or enhances its desirable properties.

### BRIEF SUMMARY OF THE INVENTION

In accordance with a first aspect of the present disclosure, an apparatus for extracting a plant extract is provided. Among other advantages, the device allows for automated extraction of a plant extract via a shearing force and incorporates solvent chambers for enhancing the desirable properties of the extract.

One embodiment of the plant press includes a container having a cavity configured to receive a plant and a plunger disposed within the cavity. The plunger may be translatable in a direction transverse to a longitudinal axis of the cavity and adapted to create a shearing force on the plant.

The plant press may further include a first chamber selectively in fluid communication with the cavity, the first chamber being configured to receive a first solvent. In an exemplary embodiment, the first chamber includes an acidic solvent. The plant press may also include a second chamber selectively in fluid communication with the cavity, the second chamber configured to receive a second solvent different than the first solvent. In an exemplary embodiment, the second chamber may include an alcoholic solvent, electrolytes, or ionized calcium.

In one embodiment, in order to increase efficiency, the plant press may include a second plunger translatable in a direction transverse to the longitudinal axis of the container and adapted to create a shearing force on the plant.

The plant press may further include a platform coupled to the container and configured to cause the container to vibrate. The plant press may also include a light emitting source for emitting light into the cavity. The light emitting source may emit at least one of UV light, visible light, infrared light, or microwave light.

The plant press may additionally include a heating or cooling device configured to modify a temperature within the cavity and a vacuum source connected to the container for reducing the pressure within the cavity, thus allowing an operator to modify the temperature within the cavity with less energy.

The plunger may be configured to twist or rotate about its axis while translating in a direction transverse to the longitudinal axis of the cavity. The plunger may include a solenoid for producing an electrical current and delivery the current to the plant. In an exemplary embodiment, a distal end of the plunger includes grooves or cutouts for enhancing the shearing force.

In another embodiment, a plant press includes a container having a cavity for receiving a plant and a plunger disposed within the cavity. The plunger being oriented such that its longitudinal axis is transverse to a longitudinal axis of the cavity and being rotatable about its longitudinal axis to create a shearing force on the plant.

In addition to being rotatable, the plunger may also be translatable in a direction transverse to the longitudinal axis of the container.

The plant press may further include first and second chambers selectively in fluid communication with the cavity of the container, the first and second chambers configured to receive a solvent. The first and second chambers may include a valve configured to transition between an open condition in which the first and second chambers are in fluid communication with the cavity and a closed condition in which the first and second chambers are in fluid isolation with the cavity. Additionally, or alternatively, the first and second chambers may include a vent configured to transition between an open condition in which the first and second chambers are in fluid communication with the cavity and a closed condition in which the first and second chambers are in fluid isolation with the cavity.

In an exemplary embodiment, a wall of the container is formed of cellulous, metal, wood, rock, plastic, or a combination thereof, for enhancing the shearing forces produced on the plant when the plant moves over a surface of the wall.

### BRIEF DESCRIPTION OF THE DRAWINGS

Various embodiments of the present disclosure are described herein with reference to the following drawings in which:

FIG. 1 is a diagrammatic representation of a plant press device in accordance with an embodiment of the disclosure;

FIG. 2 is a perspective view of a container of the plant press with the lid in the closed position.

FIG. 3 is a perspective view of the container of FIG. 2 with the lid in the open position.

FIG. 4 is a cross-section front view of the container of FIG. 3 taken along line 4-4.

FIG. 5 is a cross-section view of a plunger of the plant press.

### DETAILED DESCRIPTION

As used herein, the term “axial” means along or parallel to a longitudinal axis of the feature in which the longitudinal axis references. “Rotation” refers to rotation about the longitudinal axis, unless otherwise described. Furthermore, the term “transverse”, as used herein with respect to two axis, means the axis are not parallel to one another and, thus, will eventually transverse or extend across one another.

The plant press described herein is adapted to automate extraction of a plant extract. While the plant press is described herein in connection with extracting psychoactive resins from a cannabis plant, it will be appreciated that these concepts may be equally applicable to the extraction of different extracts from a variety of plants.

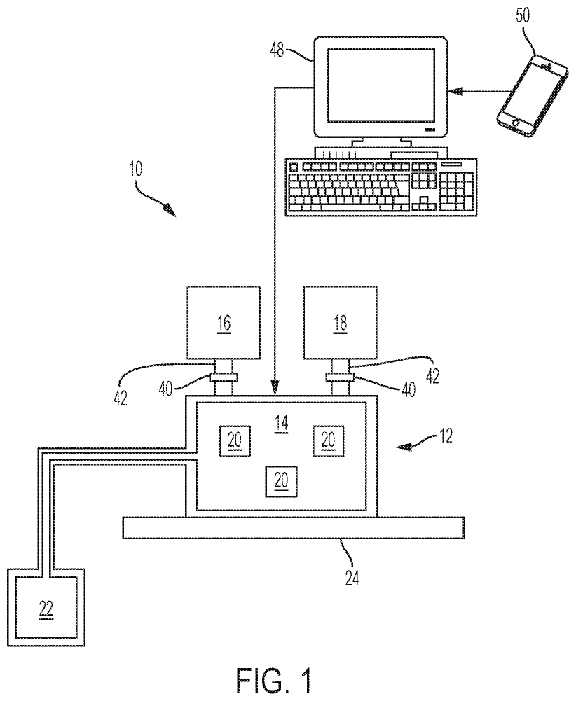

Plant press 10, diagrammatically illustrated in FIG. 1, includes a container 12 having a cavity 14, a first solvent chamber 16 and a second solvent chamber 18 selectively in fluid communication with the cavity, and one a more plungers 20 at least partially disposed within the cavity. Container 12 may be coupled to a vacuum source 22 for reducing the pressure within cavity 14 and coupled to a platform 24 adapted to shake the container, as described in further detail below.

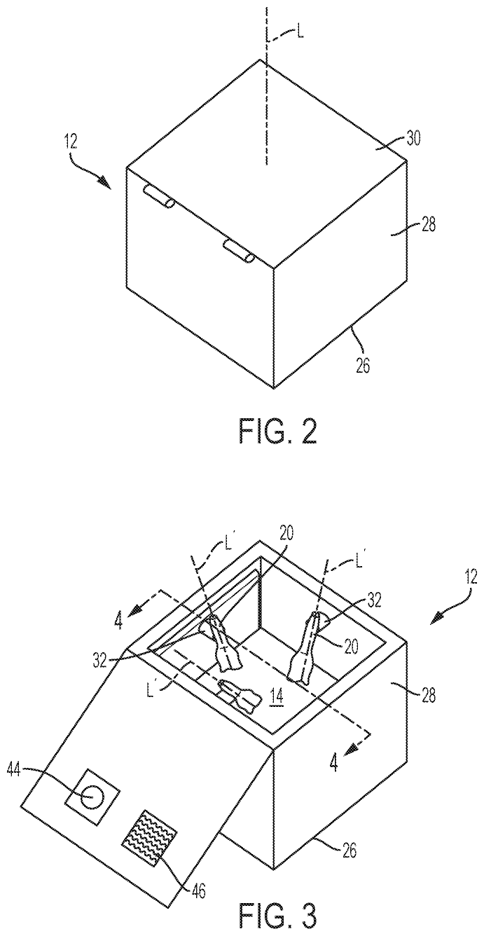

Container 12, shown in FIGS. 2-4, includes a base 26, a sidewall 28 circumscribing the base, and a lid 30, forming cavity 14 within the container 12. Lid 30 is coupled to sidewall 28, for example, via hinges such that it is transitionable from an open position (FIG. 2) to a closed position (FIG. 3). When lid 30 is in the closed position, cavity 14 is sealed from the environment. Although container 12 is illustrated as being box shaped, it will be appreciated that the container may be any shape, for example, cylindrical, spherical, or hemispherical. Container 12 is preferably formed of a material such as cellulous, metal, wood, or rock that enhances the shearing force when the plant is moved over the materials surface.

Cavity 14 includes a longitudinal axis L. One or more plungers 20, each of which have a respective longitudinal axis U, are provided within cavity 14 and oriented such that the longitudinal axis of the plunger is transverse to the longitudinal axis of cavity 14. Each one of the plungers 20 may be coupled to a motor and a drive shaft 32 for moving the plungers within cavity 14. It will be appreciated that plant press 10 may include as few as one plunger, or any number of plungers greater than one, in order to increase extraction efficiency.

Plungers 20 are adapted to move through cavity 14 in a direction that creates a shearing force and, minimizes the compression forces, on the plant. For example, plungers 20 may translate along longitudinal axis U, rotate about longitudinal axis U, simultaneously translate along longitudinal axis U and rotate about longitudinal axis U (twisting force), or otherwise move within cavity 14 to create a shearing force. As will be appreciated by one skilled in the art, compression forces generate increased friction and excess heat, which may cause heat liable compounds of the plant extract to denature and exhibit diminished desirable properties, much like the problems associated with the plant drying press. Shearing forces on the other hand, facilitate extraction and minimize heat generation.

Container 12 may be positioned on, or otherwise be coupled to, vibrating platform 24. A top surface of platform 24 may include a recess shaped to match the base 26 of container 12 or another mechanism for securing the container to the platform. Platform 24 preferably vibrates laterally, thus laterally shaking container 12 and enhancing the shearing forces applied to the plant by the plungers 20.

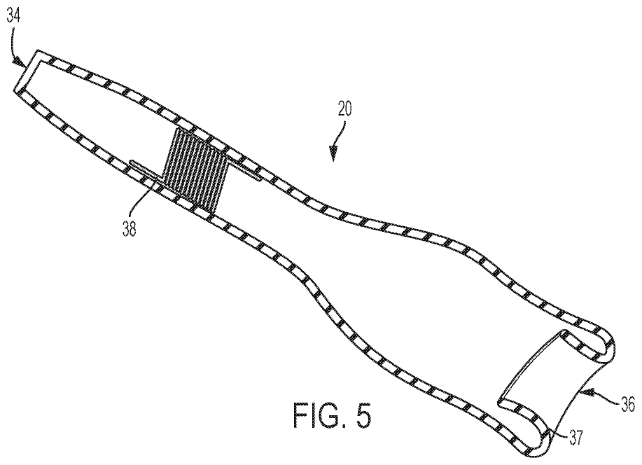

Referring to FIG. 5, each of the plungers 20 is an elongate member extending from a proximal end 34 to a distal or contacting end 36. The distal end 36 of plunger 20 may have a greater surface area than the proximal end 34, thus increasing contact area between the plunger and the plant and increasing extraction efficiency. Contact end 36 may be generally concave in shape and include a groove 37 to minimize compression forces exerted on the plant during movement of plunger 20.

Plunger 20 may also include a solenoid 38 or a battery for generating an electrical current during movement of the plunger. It has been shown that stimulating a plant with a voltage, for example, 9V causes the plant's stomata to open and facilitates extraction.

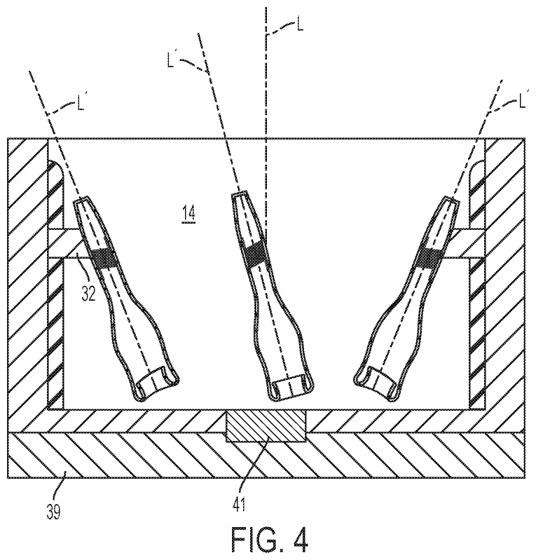

Referring back to FIG. 4, container 12 may optionally include a collection basin 39 adjacent the base 26 for collecting the plant extract. A filter 41 such as a mesh screen may be disposed between the base 26 of container 12 and the collection basin 39.

As will appreciated by one skilled in the art, ideal extraction conditions vary greatly based upon the plant. Thus, plant press 10 includes several features, further explained hereinafter with reference to FIGS. 1-4, for modifying the conditions under which extraction is performed.

First solvent chamber 16 and second solvent chamber 18 may be disposed within the sidewall 28, the lid 30, or be otherwise selectively coupled to container 12. First and second solvent chambers 16, 18 are adapted to hold one or more solvents. The first solvent chamber 16 may, for example, contain an acidic solvent while the second solvent chamber 18 may, for example, contain an alcoholic solvent, electrolytes, or ionized calcium. These solvents, however, are merely exemplary and may modified in view of the specific plant from which the user is removing an extract.

A valve 40 may be disposed between first and second solvent chambers 16, 18 and container 12. Valve 40 is transitionable from an open condition in which the first and second solvent chamber 16, 18 are in fluid communication with cavity 14 and a closed condition in which the first and second chambers are in fluid isolation with the cavity. Thus, the user can selectively regulate the quantity of solvent that is added to the slurry and at what time during the extraction process the solvent is added without having to open lid 30.

Additionally, or alternatively, a vent 42 may be disposed between first and second solvent chambers 16, 18 and container 12. Vent 42 may also be transitionable from an open condition in which the first and second 16, 18 chamber are in fluid communication with cavity 14 to a closed condition in which the first and second chambers are in fluid isolation with the cavity. In this embodiment, a fan may also be positioned within each of the first and second solvent chambers 16, 18. As such, when vent 42 is in the open condition, ionized calcium or other desirable airborne materials may be blown into cavity 14.

Container 12 may also include a light emitting source 44 attached to the lid 30 of the container for emitting light downward into cavity 14. Light emitting source 44 may emit UV light, visible light, infrared light, or microwave light to the plant during the extraction process.

Container 12 may also include a temperature regulating device 46 such as a heater or a cooling device. Temperature regulating device 46 may be disposed within the sidewall 28 or the lid 30 of container 12 for regulating the temperature within the container. In one embodiment, container 12 is optionally coupled to vacuum source 22 for altering the pressure inside of cavity 14. By reducing the pressure within cavity 14, plant press 10 is able to regulate the temperature using less energy. For example, by reducing the pressure within cavity 14, temperature regulating device 46 does not have to blow as much heat into the cavity in order to raise the temperature therein.

In a preferred embodiment, plant press 10 includes a central processing unit such as a computer 48 for controlling each of the above described components. Computer 48 may be receive a series of commands from an input device 50 such as a touch screen display, thereby specifically regulating each one of the aforementioned conditions and, even selecting the time in which the condition is performed.

Plant press 10, thus, permits the user to pre-select the conditions under which the plant is extracted and regulate the conditions with specificity. For example, a user may program plant press 10 to begin extraction at room temperature and gradually decrease the temperature by 1° C. per minute over a 30 minute extraction process and add an acidic solvent to the slurry during the 20^{th} minute. All of these conditions are automatically regulated by computer 48 such that the user does not have to open lid 30 in order to add the solvent. As a result, the desired internal environment of container 12 is maintained.

The user may use plant press 10 as follows. The user first inputs a series of controls using input device 50, specifying the desired conditions under which the plant extraction will be performed. These conditions may be tailored to the specific plant being extracted. The user may then move the lid 30 to its open position and place the plant, such as skuff, within cavity 14. After the skuff has been placed within cavity 14, the user may securely move lid 30 back to its closed position, sealing the cavity from the environment. Computer 48 will then regulate the plant extraction process according to the user's specifications. During the extraction, plungers 30 move, for example, axially and rotationally, within cavity 14, at a rate and in a direction, specified by the user in order to create a shearing force on the plant.

Upon completion of the plant extraction process, the user may remove the plant extracts from the collection basin 39 of container 12 or directly from cavity 14.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A plant press comprising:
a container including a cavity configured to receive a plant, the cavity having a longitudinal axis;
a first chamber selectively in fluid communication with the cavity, the first chamber configured to receive a first solvent; and
a plunger disposed within the cavity and being translatable in a first direction transverse to the longitudinal axis of the cavity and adapted to create a shearing force on the plant.

2. The plant press of claim 1, wherein the first chamber includes an acidic solvent.

3. The plant press of claim 1, further comprising a second chamber selectively in fluid communication the cavity, the second chamber configured to receive a second solvent different than the first solvent.

4. The plant press of claim 3, wherein the second chamber comprises an alcoholic solvent, electrolytes, or ionized calcium.

5. The plant press of claim 1, further comprising a second plunger translatable in a second direction, different than the first direction, and transverse to the longitudinal axis of the container.

6. The plant press of claim 1, further comprising a platform coupled the container and configured to cause the container to vibrate.

7. The plant press of claim 1, further comprising a light emitting source for emitting light into the cavity.

8. The plant press of claim 7, wherein the light emitting source emits at least one of UV light, visible light, infrared light, or microwave light.

9. The plant press of claim 1, further comprising a vacuum source connected to the container for reducing the pressure within the cavity.

10. The plant press of claim 1, wherein the plunger includes a solenoid for producing an electrical current.

11. The plant press of claim 1, wherein the plunger is further configured to rotate.

12. The plant press of claim 1, further comprising a heating or cooling device for modifying a temperature within the cavity.

13. A plant press comprising:
a container including a cavity for receiving a plant, the cavity having a longitudinal axis;
first and second chambers selectively in fluid communication with the cavity of the container, the first and second chambers configured to receive a solvent; and
a plunger disposed within the cavity, the plunger having a longitudinal axis transverse to the longitudinal axis of the cavity, wherein the plunger is rotatable about its longitudinal axis and configured to create a shearing force on the plant.

14. The plant press of claim 13, wherein the plunger is translatable in a direction transverse to the longitudinal axis of the container.

15. The plant press of claim 13, further comprising a valve disposed between the first and second chambers and the cavity, the valve being transitionable between an open condition in which the first and second chambers are in fluid communication with the cavity and a closed condition in which the first and second chambers are in fluid isolation with the cavity.

16. The plant press of claim 13, further comprising a vent disposed between the first and second chambers and the cavity, the vent being transitionable between an open condition in which the first and second chambers are in fluid communication with the cavity and a closed condition in which the first and second chambers are in fluid isolation with the cavity.

17. The plant press of claim 13, wherein a wall of the container is formed of cellulous, metal, wood, rock, or plastic, or a combination thereof.

18. The plant press of claim 13, wherein a distal end of the plunger is concave for reducing compression forces.

19. The plant press of claim 13, wherein a distal end of the plunger includes grooves or cutouts for enhancing the shearing force.

20. A method for extracting a plant extract from a plant, comprising:
positioning a plant material within a cavity of a container;
sealing the cavity from an environment;
applying a solvent to the plant material;
applying a sheering force to the plant material; and
collecting plant extract extracted from the plant,
wherein the steps of applying the solvent to the plant material and applying the sheering force to the plant material are carried out when the cavity is sealed from the environment.
